(19) United States
(12) Patent Application Publication
(10) Pub. No.: US 2014/0120176 A1
(43) Pub. Date: May 1, 2014

(54) Oxygenated Water and Uses Thereof

(71) Applicants: Michael Thorp, Oslo (NO); Mahmood Amiry-Moghaddam, Oslo (NO); Jan Økern, Nesoya (NO); Laura Maria Azzurra Camassa, Oslo (NO)

(72) Inventors: Michael Thorp, Oslo (NO); Mahmood Amiry-Moghaddam, Oslo (NO); Jan Økern, Nesoya (NO); Laura Maria Azzurra Camassa, Oslo (NO)

(21) Appl. No.: 14/111,736

(22) PCT Filed: Apr. 16, 2012
(86) PCT No.: PCT/EP2012/056948

§ 371 Date: Jan. 16, 2014

(30) Foreign Application Priority Data
Apr. 14, 2011 (NO) 20110574
Apr. 14, 2011 (US) 61475492

(51) Int. Cl. A61K 33/00; A61Q 19/00; A61K 8/19

(52) U.S. Cl. CPC A61K 33/00; A61K 8/19; A61Q 19/00
USPC 424/600; 435/1.1; 435/383; 435/404

(57) ABSTRACT

The invention relates to solutions carrying high levels of oxygen, e.g. to aqueous solutions having an oxygen concentration of at least 30 mg/L, especially at least 70 mg/L, and to uses thereof. The aqueous solutions contain colloidal minerals which help stabilise the high oxygen levels. The super-oxygenated fluids may be used to affect the viability, growth and maintenance of cells, tissues and organs.

The present invention relates to solutions having a high content of oxygen and the use thereof. More specifically, the present invention relates to solutions having a high content of oxygen, and their use for affecting the viability, growth and/or maintenance of cells, tissues and organs, in different environments.

Life on earth is dependent on the ability of water to dissolve oxygen. The oxygen level in water is dependent on the pressure, the temperature, the oxygen level in the surrounding atmosphere, any oxygen consuming or oxygen producing activity (e.g. animal or plant cells present therein), and the dissolution rate. The dissolution rate is again dependent on the contact surface between the water and source of oxygen, such as air.

For applications where oxygen present in the water is consumed, such as e.g. an aquarium, cell or tissue cultures and the like, oxygen has traditionally been supplied by bubbling air trough the water or aqueous medium, or by keeping the aqueous medium in an incubator with increased oxygen partial pressure. Several applications for increasing the contact surface between the air and water/aqueous medium, such as by creation of micro bubbles, have been developed. To improve the rate of adsorption/dissolution, air may be substituted by oxygen or oxygenated air.

The level of oxygen in a liquid, such as water or an aqueous medium is, however, dependent on the solubility of oxygen in the liquid. If the level of dissolved oxygen in normal water is higher than the solubility, the surplus oxygen will after a while escape to the surrounding atmosphere or form bubbles. At 25° C. at atmospheric pressure, the solubility of oxygen is about 7 mg/L. Under laboratory conditions it is possible to produce water super saturated with oxygen, having an oxygen content of 28-30 mg/L. The super saturated water is, however, unstable and will rapidly release oxygen until equilibrium corresponding to about 7 mg/L is reached.

Efforts have been made to increase the level of oxygen in liquids above the “normal” solubility. WO 02/26367 of Geir Corporation relates to an apparatus and method for increasing oxygen levels in a liquid, to form a stable oxygenated liquid where oxygen is kept dissolved an oxygen level being substantially higher than what is expected from “normal” liquid.

WO 2010/077962, also of Geir Corporation, relates to improvements in oxygenation of a liquid. According to both WO 02/26367 and WO 2010/077962, the oxygen level in a liquid, such as water, may be substantially increased by introduction of colloidal minerals into the liquid, and dissolution of oxygen and stabilization of the solution in a series of method steps. Tests have shown that the stably dissolved oxygen content of water prepared by the method of the mentioned patents may be higher than 70 mg/L or about 10 times the oxygen level in natural water, or higher.

Availability of sufficient oxygen is a critical factor for survival and viability of many cell types, both in vivo and in vitro. The required oxygen concentration for survival and viability may, however, vary substantially between species. For some animals most or all oxygen is delivered directly to the individual cells from the surroundings, whereas other animals have a blood system, or the like, for delivery of i.a. oxygen to all or most cells. In mammalians, most cells receive the oxygen needed directly or indirectly from blood circulation.

The oxygen demand and the dependency of oxygen for survival vary dramatically from cell type to cell type. Brain cells, especially neurons, are extremely oxygen dependent or aerobic cells and will be seriously injured or even die after a few minutes without sufficient supply of oxygen. Other cell types may, on the other side, survive for hours without supply of oxygen. All mammals do, however, need oxygen for growth and viability.

The oxygen content of other liquids at ambient temperature is varying dependent on the liquid in question. Both WO 02/26367 and WO 2010/077962 describe oxygenation of other liquids than water, such as oils. For some applications, oil or an oil based medium, may be used as an oxygen carrier.

Aqueous media has at equilibrium at ambient temperature (about 20° C.) an oxygen content of about 7 mg/L, somewhat dependent on the composition of the media. When culturing oxygen dependent cells in vitro, the medium used is oxygenated by means of bubbling air though the medium normally in a separate oxygenation unit.

Even if there have been developed solutions for obtaining larger surface of contact between the air and the liquid, the amount of oxygen in the water is dependent on the factors above, and the rate of oxygenation.

According to a first aspect, the present invention relates to a liquid medium for providing oxygen to cells, tissues or organs, wherein the medium, comprises a liquid oxygenated by:
introducing a pressurized liquid into a piping network to form a flow stream,
if necessary, adding colloidal minerals to a desired concentration to the flow stream,
injecting gaseous oxygen into the flow stream to produce a mixture of liquid and oxygen bubbles,
providing a linear flow accelerator including a venturi and a magnet adjacent to the venturi and aligned along the venturi for applying a magnetic field of desired field strength across the venturi, and
passing the flowing mixture of liquid and gaseous oxygen bubbles through the linear flow accelerator to accelerate the flowing mixture and to subsequently decelerate the flowing mixture to subsonic speed to break up the gaseous oxygen bubbles.

The invention also extends to the use of such a liquid medium for providing oxygen to cells, tissues or organs.

Oxygenation using the above described method makes it possible to produce an oxygenated liquid having a stable oxygen content that is substantially higher than what is expected from the solubility of oxygen in the liquid at equilibrium with the surroundings. The above described method produces stable, oxygen-containing liquids comprising colloidal minerals, typically at a level of about 10 to about 50 ppm. When the liquid is water, the solubility of oxygen is increased from about 7 mg/L to 50, 60, 70 mg/L or more, and the oxygen content is substantially stable in a cooled bottle for weeks. In vitro tests do, however, show improved survival and viability for cells kept in a culture media comprising the present medium, results that strongly indicate that the oxygen therein is made available.

According to one embodiment, the liquid is water. According to a specific embodiment, the oxygen content of the water is 10 mg/L or higher. The oxygen content may, however, be higher, such as above 15 mg/L, above 20 mg/L, above 30 mg/L, above 50 mg/L, above 60 mg/L or above 70 mg/L.

According to one embodiment, the liquid is an oil. The oxygen content may be as defined above, e.g. 10 mg/L or higher, or above 30 mg/L.

According to a second aspect, the invention relates to a method for promoting growth of aerobic cells at the expense of anaerobic cells, wherein the method comprises the step of delivering an oxygenated liquid medium as described above, to cells, tissues, organs or the like. In a related aspect, the invention provides the use of an oxygenated aqueous solution as defined herein for promoting growth of aerobic cells at the expense of anaerobic cells. The invention also provides a method for promoting growth of aerobic cells at the expense of anaerobic cells wherein an oxygenated liquid medium as defined herein is used.

According to a third aspect, the invention relates to adding an aqueous medium as described above, to a compost to promote aerobe degradation processes.

According to a fourth aspect, the invention relates to an oxygenated aqueous solution for culturing cells, tissue, maintaining cells, tissues or organs, or the like, where the solution comprises solutes traditionally added to such a solution, wherein all or parts of the water in the solution is substituted by oxygenated water having an oxygen content of more than 10 mg/L, e.g. more than 20 mg/L or more than 30 mg/L. The oxygen content may, however, be higher, such as above 50 mg/L, above 60 mg/L or above 70 mg/L.

In a related aspect, the invention provides the use of an aqueous solution for culturing cells or tissues, or for maintaining cells, tissues or organs, wherein the aqueous solution comprises oxygenated water comprising colloidal minerals, said oxygenated water having an oxygen content of more than 20 mg/L.

According to a fifth aspect, the invention relates to a method for culturing or maintaining cells, tissue, organs or the like, where the cells, tissues, organs or the like are kept in an aqueous medium comprising traditional solutes for culturing or maintaining viability, wherein all or parts of the water in the aqueous medium is substituted by oxygenated water having an oxygen content of more than 10 mg/L, e.g. more than 20 mg/L or more than 30 mg/L. The oxygen content may, however, be higher, such as above 50 mg/L, above 60 mg/L or above 70 mg/L. The oxygenated water preferably comprises colloidal minerals.

According to a sixth aspect, the present invention relates to a medium for in vitro culturing of mammal cells, comprising conventional constituents for a culture medium, characterised in additionally comprising at least 10% of the medium described above, where the liquid is water. In a related aspect the invention provides the use of such a medium for the in vitro culturing of mammal cells, as well as providing a method for the in vitro culturing of mammal cells using the said medium.

According to a seventh aspect, the invention relates to a physiologically acceptable buffer for (e.g. suitable for) the in vivo treatment of a tissue where the oxygen supply is impaired, wherein the buffer comprises highly oxygenated water having an oxygen content of more than 10 mg/L, e.g. more than 20 mg/L or more than 30 mg/L. The oxygen content may, however, be higher, such as above 50 mg/L, above 60 mg/L or above 70 mg/L. The physiologically acceptable buffer will preferably comprises conventional additives.

According to an eighth aspect, the invention relates to a method for treating a deoxygenated tissue or for the in vivo treatment of a tissue where the oxygen supply is impaired, wherein a physiological acceptable buffer having an oxygen content of more than 10 mg/L, e.g. more than 20 mg/L or more than 30 mg/L, is supplied to the tissue. The oxygen content may, however, be even higher, such as above 50 mg/L, above 60 mg/L or above 70 mg/L. The invention also extends to the use of oxygenated aqueous solutions as defined herein for the in vivo treatment of a tissue where the oxygen supply is impaired, or for treating a deoxygenated tissue.

According to a ninth aspect, the invention relates to a method for treatment or amelioration of a medical condition where a reduced level of oxygen in a tissue, organ or the like is implicated, wherein a physiologically acceptable preparation including water having an oxygen content of more than 10 mg/L, e.g. more than 30 mg/L, is administered to the tissue, organ or the like. The oxygen content may, however, be even higher, such as above 50 mg/L, above 60 mg/L or above 70 mg/L.

In a related embodiment the present invention provides a method of treatment or amelioration of a medical condition where reduced levels of oxygen in a tissue, organ or the like is implicated, the method comprising administering to a subject in need thereof a preparation comprising an oxygenated liquid (e.g. water or an oil) comprising colloidal minerals and having a stable oxygen content which is higher than the equilibrium oxygen content of the liquid alone. The invention also provides an oxygenated liquid as defined herein for use in such methods as well as providing the use of an oxygenated liquid as defined herein in the preparation of a medicament for use in such methods. The medical condition to be treated is preferably selected from stroke, migraine headache, cerebral haemorrhage, and any other neurological condition where neuroprotective agents are indicated; myocardial infarction, angina pectoris or any other cardiac disorder caused by reduced oxygen supply; a clinical condition where oxygen supply is needed, e.g. corneal oxygen deficiency; a condition where accelerated wound healing is desired, e.g. burn, diabetic ulcer or common wound; and a condition where the presence of anaerobic bacteria is a major concern.

The present invention also extends to the use of oxygenated liquids comprising colloidal minerals and having a stable oxygen content which is higher than the equilibrium oxygen content of the liquid alone as defined herein in therapy, e.g. their use as medicaments.

According to a tenth aspect, the invention relates to a cosmetic preparation, comprising conventional constituents for cosmetics, wherein all or parts of the liquid in the cosmetic preparation are substituted by a liquid medium as described above, preferably wherein the liquid medium is oxygenated water as defined herein. The invention further relates to a method of cosmetic treatment, the method comprising administering a liquid preparation (e.g. a topical preparation) to the skin of a subject, wherein the preparation comprises an oxygenated liquid comprising colloidal minerals and having a stable oxygen content which is higher than the equilibrium oxygen content of the liquid alone.

Further aspects of the invention are found in the following numbered embodiments:

1. A liquid medium for providing oxygen to cells, tissues or organs, wherein the medium, comprises a liquid oxygenated by:
a. introducing a pressurized liquid into a piping network to form a flow stream,
b. adding colloidal minerals to a desired concentration to the flow stream, if colloidal minerals are naturally present in the addition of such minerals may be reduced or even omitted,
c. injecting gaseous oxygen into the flow stream to produce a mixture of liquid and oxygen bubbles,
d. providing a linear flow accelerator including a venturi and a magnet adjacent to the venturi and aligned along the venturi for applying a magnetic field of desired field strength across the venturi, and
e. passing the flowing mixture of liquid and gaseous oxygen bubbles through the linear flow accelerator to accelerate the flowing mixture and to subsequently decelerate the flowing mixture to subsonic speed to break up the gaseous oxygen bubbles.

2. The liquid medium according to numbered embodiment 1, wherein the liquid is water.

3. The medium according to numbered embodiment 2, wherein the oxygen content of the water is 30 mg/L or higher, e.g. 50 mg/L or higher.

4. The liquid medium according to numbered embodiment 1, wherein the liquid is an oil.

5. A method for promoting growth of aerobic cells at the expense of anaerobic cells, wherein the method comprises the step of delivering a liquid medium according to numbered embodiment 1 to cells, tissues, organs or the like.

6. The method of numbered embodiment 5, wherein a liquid medium according to numbered embodiment 2 is added to a compost to promote aerobe degradation processes.

7. An oxygenated aqueous solution for culturing cells, tissue, maintaining cells, tissues or organs, or the like, where the solution comprises solutes traditional traditionally added to such a solution, wherein all or parts of the water in the solution is substituted by oxygenated water having an oxygen content of more than 30 mg/L, e.g. more than 50 mg/L.

8. A method for culturing or maintaining cells, tissue, organs or the like, where the cells, tissues, organs or the like are kept in an aqueous medium comprising traditional solutes for culturing or maintaining viability, wherein all or parts of the water in the aqueous medium is substituted by oxygenated water having an oxygen content of more than 30 mg/L, e.g. more than 50 mg/L.

9. A medium for in vitro culturing of mammal cells, comprising conventional constituents for a culture medium, characterised in additionally comprising at least 10% of the medium according to claim 1 where the liquid is water.

10. A physiologically acceptable buffer for in vivo treatment of a tissue where the oxygen supply is impaired, wherein the buffer comprises highly oxygenated water having an oxygen content of more than 30 mg O₂/L, e.g. more than 50 mg O₂/L.

11. The physiological acceptable buffer of numbered embodiment 10, wherein all of the water therein has an oxygen content of more than 30 mg O₂/L, e.g. more than 50 mg O₂/L.

12. A method for treating a deoxygenated tissue, wherein a physiological acceptable buffer having an oxygen content of more than 30 mg O₂/L, e.g. more than 50 mg O₂/L, is supplied to the tissue.

13. A method for treatment or amelioration of a medical condition where reduced level in a tissue, organ or the like is implicated, wherein a physiologically acceptable preparation including water having an oxygen content of more than 30 mg/L, e.g. more than 50 mg/L, is administered to the tissue, organ or the like.

14. A cosmetic preparation, comprising conventional constituents for cosmetics, wherein all or parts of the liquid in the cosmetic preparation is substituted by a liquid medium according to numbered embodiment 1.

Liquids, such as water, having a high oxygen level are known from i.a. the above mentioned WO 02/26367 and WO 2010/077962. The liquids having a high level of oxygen used according to the present invention are preferably produced according to the methods and by means of the devices described therein. Due to the process for the super oxygenation of liquids, the oxygenated liquid comprises 10 to 50 ppm colloidal minerals, in addition to other trace elements present in the water used for the production.

As described in WO 02/26367 and WO 2010/077962 (the entire contents of which are incorporated herein by reference), suitable colloidal minerals are characterized by having electrostatic adsorption of ions to the surface of a colloidal particle. This adsorption creates a primary adsorption layer that in turn creates a substantial adsorption layer at the surface of the colloidal particle. Thus, the colloidal minerals provide electrostatic surface ion absorption characteristics that can enhance the ability of the water to absorb and retain injected oxygen. The amount and types of colloidal minerals added to the water will depend on the requirements of a given application.

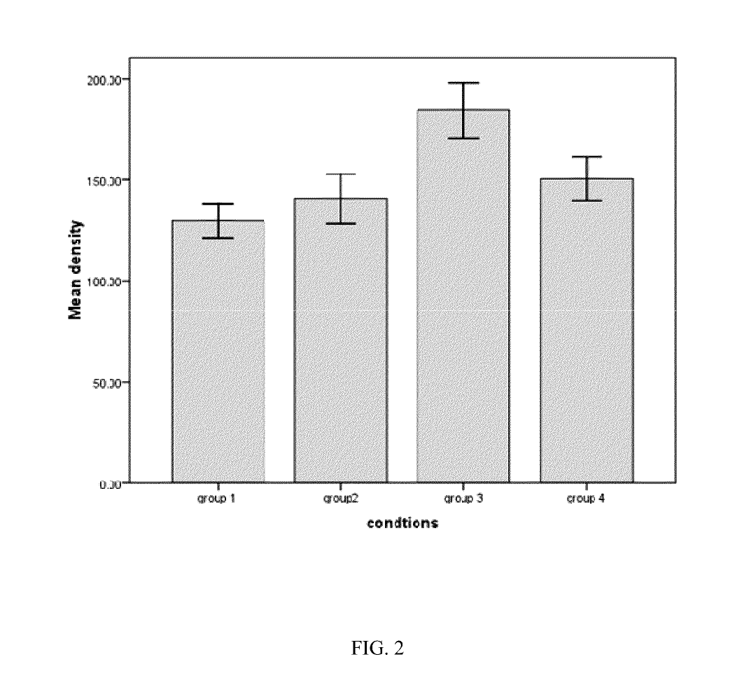

Examples of suitable colloidal minerals include aluminium, sulphur, iron and fluoride. Examples of specific mineral compositions that may be used include those produced by The Rockland Corporation (Tulsa, Okla., USA) under the trademark Body Booster, and by TRC Nutritional Laboratories, Inc. (Tulsa, Okla., USA) under the trademark TRC Minerals®. A suitable formulation of 77LPPM TRC Minerals is set out in the Table of FIG. 2 of WO 2010/077962.

The liquid having a high level of oxygen produced as indicated above, is stable and may be stored in tanks or be bottled between production and use for e.g. the purpose of the present invention.

As used herein, when using the term cells, also tissues and organs are encompassed, and vice versa, if otherwise is not clearly stated or clearly apparent from the context.

The oxygenated liquids used in the present invention may be produced by means of a process mainly as described in U.S. Pat. No. 6,821,438, (the entire content of which is incorporated herein by reference). The method of U.S. Pat. No. '438 comprises the steps of:
introducing a pressurized liquid into a piping network to form a flow stream,
adding colloidal minerals to a desired concentration to the flow stream, if colloidal minerals are naturally present in the flow stream the addition of such minerals may be reduced or even omitted,
injecting gaseous oxygen into the flow stream to produce a mixture of liquid and oxygen bubbles,
providing a linear flow accelerator including a venturi and a magnet adjacent to the venturi and aligned along the venturi for applying a magnetic field of desired field strength across the venturi, and
passing the flowing mixture of liquid and gaseous oxygen bubbles through the linear flow accelerator to accelerate the flowing mixture and to subsequently decelerate the flowing mixture to subsonic speed to break up the gaseous oxygen bubbles.

After separation of liquid and oxygen bubbles, the liquid is filled into containers, such as bottles or other convenient containers, until use thereof.

For many of the presently preferred applications, water is the liquid that is oxygenated. For other applications non-aqueous liquids, such as oils, may be oxygenated.

The invention will now be further described with reference to the following non-limiting Examples and Figures, in which:

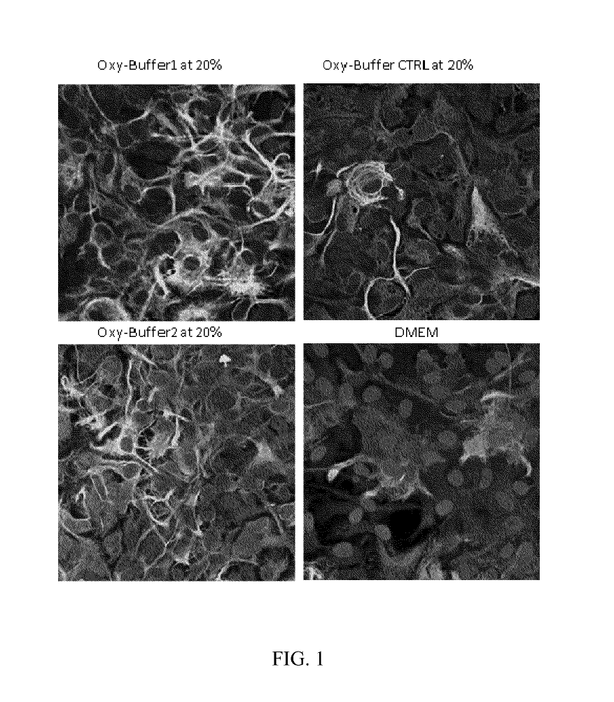

FIG. 1 shows confocal images of astrocytes grown in 4 different conditions. GFAP (glial febrillary protein) is in green, aquapotin-4 (AQP4; membrane protein expressed in astrocytes) is in red and DAPI-nuclei are in blue. Scale Bar 50 μm; Object magnification: 40×.

FIG. 2 is a graph showing the mean of the density of cells per area for each condition. p≦0.05 and the error bars represent the 95% CI (Interval of Confidence). The 4 groups analysed are: group 1: astrocytes grown with Oxy-Buffer CTRL; group 2: astrocytes grown in the usual media; group 3: astrocytes grown with Oxy-Buffer2; and group 4: astrocytes grown with Oxy-Buffer unstable in O₂ content (Oxy-Buffer1 at 20%). Note that addition of Oxy-Buffer with unstable oxygen content (group 4) or Control Oxy-Buffer (with normal colloidals but without oxygen) didn't have a significant effect on the cell density compared to the control (DMEM).

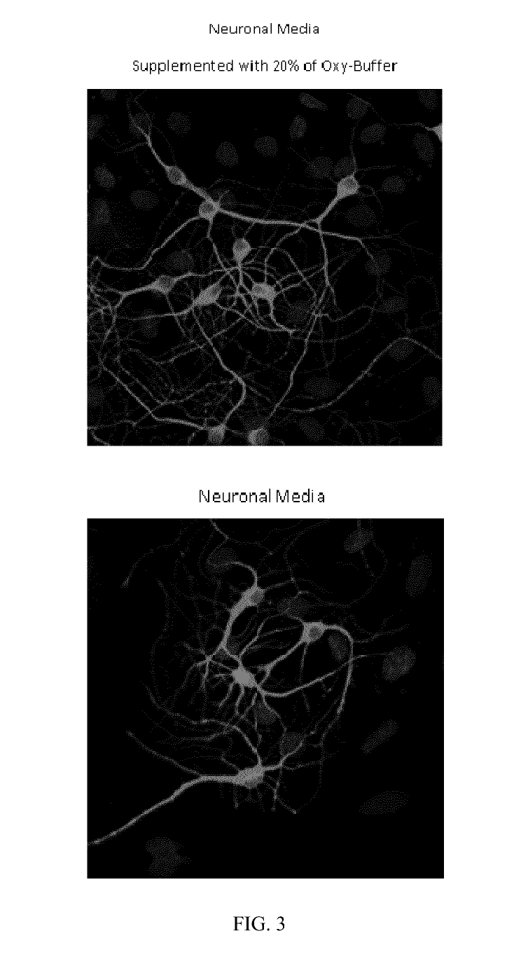

FIG. 3 shows confocal images of neurons stained with βIII-tubulin (a neuronal marker) in red and DAPI stained nuclei in blue. Note relatively higher number of neuronal processes in the group with supplement of Buffer with high content of oxygen. Scale Bar 50 μm; Object magnification: 40×.

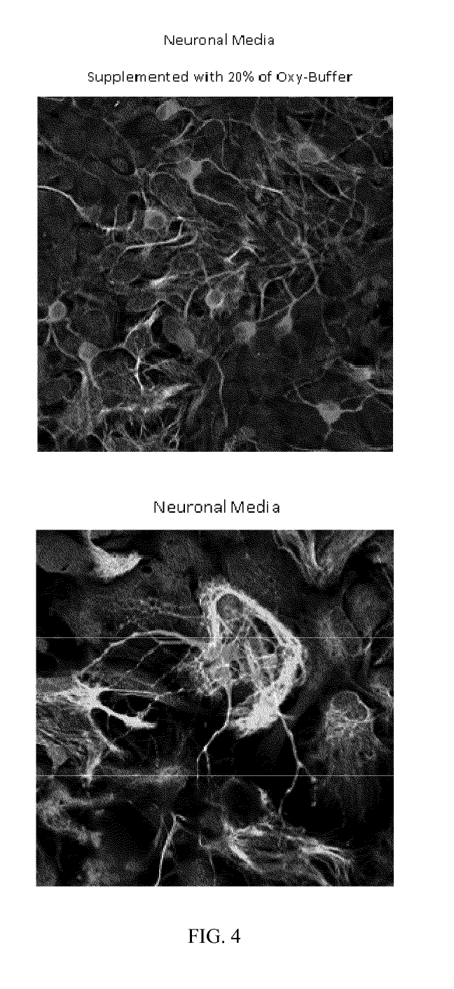

FIG. 4 shows Confocal images of neurons and astrocytes stained respectively with βIII-tubulin in red and GFAP in green, DAPI-stained nuclei are in blue. Scale Bar 50 μm; Object magnification: 40×.

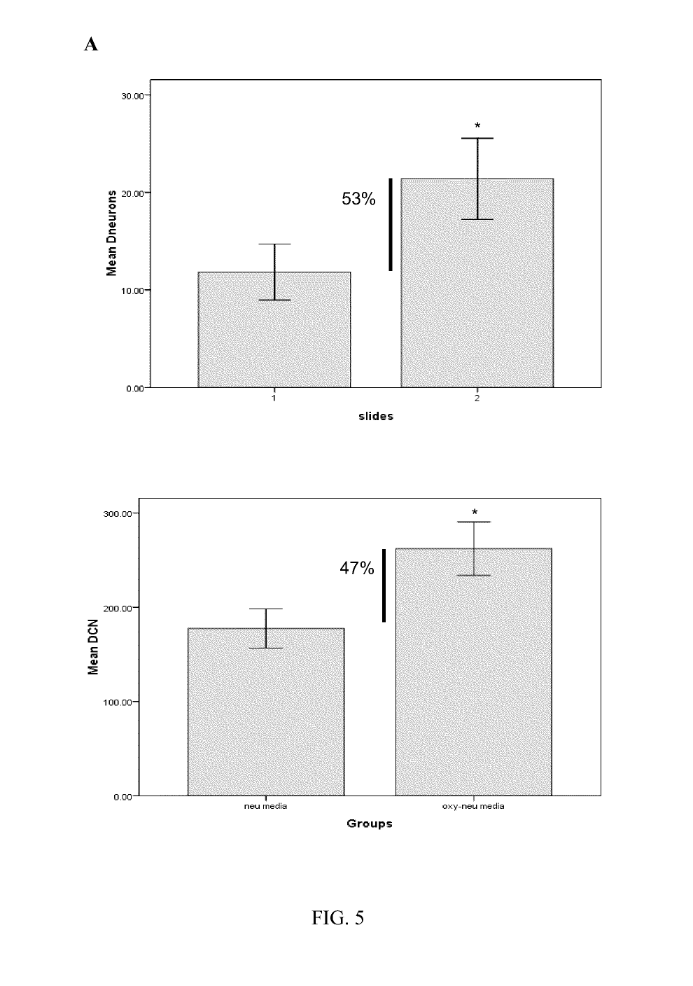

FIG. 5 shows histogram A which illustrates the mean density of neurons grown in the media supplemented with Buffer with high content of oxygen. Condition 1 denotes cells grown in normal neuronal media whereas condition 2 denotes cells grown in neuronal media supplemented with 20% of Oxy-water. The graph B shows an increase in the density of the astrocytes in the co-culture. Error bars on the graphs denote 95% CI.

In the Examples, water having high and stable oxygen content at about 70 mg/L oxygen was prepared according to WO 2010/077962. A buffer with high content of oxygen having the composition of Table 1, was prepared using the oxygenated water:

TABLE 1

| Constituent | Concentration (mM) |
|---|---|
| NaCl | 137 |
| KCl | 2.7 |
| Na₂HPO₄ | 10 |
| KH₂PO₄ | 1.76 |

### EXAMPLE 1

### Primary Culture of Astrocytes and Experiment with Buffer with High Content of Oxygen

Primary cultures of cortical astrocytes from rat newborn (1-2 days) were prepared according to the modified method of McCarthy and deVellis (McCarthy et al., J. Cell Biol. (1980) 85(3):890-902). After decapitation and removal of meninges, cerebral cortices were collected in Falcon tubes containing Dulbecco's modified Eagle's medium (DMEM)-glutamax (4500 mg/L of glucose) with 10% foetal bovine serum (FBS) and penicillin/streptomycin (100 U/mL and 100 μg/mL, respectively). All the products were purchased from Gibco-Invitrogen. After mechanical dissociation and passage through cell strainers (70 μm Nylon from BD Falcon), the cells were plated in poly-D-lysine (Sigma) coated 75 cm² flasks (Corning® Flasks) containing 15 mL of DMEM-glutamax, 10% FBS and pen/strep (Gibco-Invitrogen). The flasks were maintained in an incubator with humidified atmosphere at 37° C. containing 5% CO₂. The culture medium was changed every 2 days. In order to detach the microglia and precursor cells grown on top of the layer of protoplasmic astrocytes, the flasks were gently shaken for 2 min at each change of the medium.

Successively, the astrocytes were seeded at the concentration of 2×10⁴ cells/ml on cover slips previously treated with poly-D-lysine. At day 1, the initial media were substituted with the experimental media. The media were substituted with fresh media every 2 days for one week. After one week the cells were fixed in 4% PFA in phosphate buffer, stained by immunofluorescence technique, images taken by a camera connected to a confocal microscope. The cells were then counted followed by statistical analysis.

### Experimental Groups

The fresh primary astrocytes (3 different plates in each group) were treated with 4 different media for one week as described above. The following experimental media were used:

1) Normal media (DMEM) used for the astrocytes previously described;

2) DMEM supplemented with 20% of buffer made with water which contains just colloidal particles but no oxygen;

3) DMEM supplemented with 20% of Buffer with high content of oxygen (from oxygenated water with a high and stable oxygen content); and

(4) DMEM supplemented with 20% volume of Buffer with high content of oxygen (from a series with unstable oxygen content).

### EXAMPLE 2

### Primary Co-Culture of Hippocampal Neurons and Astrocytes

Primary culture of hippocampal neurons from rat newborn (1-2 days) was prepared as follows. After decapitation (new born Wistar rat) and removal of meninges, the brain was dissected in two hemispheres using a scalpel and forceps. The hippocampus were located and isolated from the rest of the hemispheres. Each hippocampus was dived into 4-5 pieces and kept in an ice-cold solution composed of Hanks (HyClone), HEPES (Sigma) and 20% Foetal bovine serum (FBS). After successive washing procedures, the hippocampi was dissociated and digested to arrive at a solution containing single cells.

The cells were then applied on glass cover slips, previously treated with Matrigel™ (Sigma) and kept in the incubator to avoid the matrigel polymerization.

The cells were grown in a 24 multiwell (Corning) with a neuronal media, with the following composition:

Stock solution made of MEM (Gibco, Invitrogen),

4.5 g/L Glucose,

200 mg/L NaHCO₃ (Sigma) and

100 mg/L Transferrin (Calbiochem) and 10% FBS,

0.2 M L-Glutamine, Insulin and a neuronal supplement B27 (Gibco).

From the same preparation, another 24 multiwell with cells were grown with the neuronal media with the same composition described above, plus the 20% of Buffer with high content of oxygen.

After 5 days the cells where fixed in 4% PFA in phosphate buffer and experiments of immunofluorescence were conducted on them, followed by statistical analysis.

### Immunofluorescence—Statistical Analysis

Astrocytes and neurons were fixed phosphate buffer formaldehyde 4% (PFA4%) for 30 min, rinsed in Phosphate Buffer Saline (PBS) solution, and permeabilized for 10 min with 0.1% Triton X-100 in PBS. After blocking with 2% bovine serum albumin (BSA) in PBS, the samples were incubated overnight with primary antibodies. The primary antibodies were used at the following dilution:

Chicken anti-GFAP (Glial Fibrillary Acidic Protein; marker for astrocytes), (Covance Nordic BioSite),
1:1000; goat anti-AQP4 (s.c. 9888, Santa Cruz, Inc.), 1:300; protein, anti-β III tubulin monoclonal-rabbit (Covance Nordic BioSite) 1:100.

After washing in PBS, the samples were incubated with the following secondary antibodies diluted 1:1000 in PBS for 1 hour:

Cy2 donkey-anti chicken, Alexa 594 donkey anti-goat (Invitrogen), and Cy3 donkey anti-rabbit (Jackson Immuno Research Laboratories, Inc.). After washing in PBS, the cells were mounted with mounting solution Prolong® Gold antifade reagent with DAPI (marker for the nuclei; Invitrogen, Molecular Probes) and analyzed at Leica confocal microscopy, with an oil objective 40×.

10 images were taken from each group (n=3). Cell count per area (density cells/mm²) for each image was made based on the DAPI-nuclei staining.

The cell count and density values relatives for each group were analysed in the statistical program SPSS with Anova and 95% of Confidence Interval. All images were taken and analysis performed in blinded manner.

### Results and Conclusions

Primary Astrocytes:

The confocal pictures shown in FIG. 1, show primary culture of astrocytes in 4 different groups as described above:

1) normal medium (DMEM),

2) media supplemented at the 20% with Buffer with high content of oxygen without O2 (Buffer with high content of oxygen CTRL at 20%),

3) media supplemented with 20% Buffer with high content of oxygen with stable O2 content (Buffer with high content of oxygen2 at 20%), and

4) media supplemented with 20% Buffer with high content of oxygen with unstable O2 content (Buffer with high content of oxygen1 at 20%). The images show astrocytes with normal shape and GFAP (green) expression.

The density and cell count values relatives for each group were analysed with the statistical program SPSS (Anova) and 95% of Confidence Interval.

The results show that the group grown in the media supplemented with 20% Buffer with high content of oxygen with stable oxygen content have a cell density which is more than 30% higher than the other groups.

The graph illustrated in FIG. 2, shows the mean of the density of cells per area for each condition. p<0.05 and the error bars represent the 95% CI (Confidence interval). The 4 groups analysed are:

group 1: astrocytes grown with Buffer with high content of oxygen CTRL;

group 2: astrocytes grown in the usual media;

group 3: astrocytes grown with Buffer with high content of oxygen2;

group 4: astrocytes grown with Buffer with high content of oxygen unstable in O2 content (Buffer with high content of oxygen1 at 20%).

Note that addition of Buffer with high content of oxygen with unstable oxygen content (group 4) or Control Buffer with high content of oxygen (with normal colloidals but without oxygen) didn't have a significant effect on the cell density compared to the control (DMEM).

Primary Hippocampal Astrocyte-Neuron Co-Culture:

FIGS. 3 and 4 illustrate immunofluorescence images representative of hippocampus neuronal cultures grown with neuronal media supplemented with 20% of Buffer with high content of oxygen and neurons grown with the normal neuronal media without 20% of buffer with normal water. The two images of FIG. 3 visualizes neurons in red (β III tubulin) while in the two images of FIG. 4 visualize both neurons and astrocytes (GFAP, green).

The cell density for each group was analysed in SPSS with Anova and 95% of Confidence Interval. The results are illustrated in FIG. 5 showing histograms.

On the X axis the two conditions of growing and on the Y axis the mean of the cell density (cells/mm²) are shown. Histograms A of FIG. 5 show the mean density of neurons grown in the media supplemented with Buffer with high content of oxygen is 53% higher than the neurons grown in the control media. The graph in B shows an increase in the density of the astrocytes in the co-culture. Density of astrocytes grown in the media with addition of Buffer with high content of oxygen is 47% higher than the density of astrocytes grown in the control media.

### CONCLUSIONS

The data show that addition of buffers with high content of oxygen to the culture medium leads to

1) Significantly higher density of astrocytes (32-47%) and neurons (53%),

2) neurons grown in the medium with addition of buffer with high content of oxygen reveal increased growth of neurites and improved viability,

3) Buffer with high content of oxygen could be used as supplement to the culture medium for brain cells and most probably for primary cultures from other tissue.

### Discussion

The examples above clearly illustrate the improved growth and viability of astrocytes and neurites, by supplying highly oxygenated water to the growth medium.

Neurons are primary examples of cells that are difficult to keep alive and relatively difficult to get to grow in vitro. There is good reason to assume that the viability and growth for other cells having the same or corresponding need for oxygen for growth and viability in vitro, will improve correspondingly.

Possible indications for the solutions having a high content of oxygen, are:
As a supplement to all culture media for culturing aerobic cells, or tissues,
As a supplement to all media for isolation and growth of stem cells, both in clinical and laboratory settings,
As a supplement to media where acutely dissociated cells are kept without culturing,
As a supplement to the medium where tissue or organs are kept under, i.e. awaiting, surgery.

From the results above it may also be concluded that the highly oxygenated water can be used clinically for conditions where the oxygen supply to a tissue is impaired. A non-exhaustive list of possible indications that may be treated with a physiological acceptable solution comprising the highly oxygenated water as described herein, comprises the following indications:
conditions such as stroke, migraine headache, cerebral haemorrhage, and any other neurological condition where neuroprotective agents are indicated,
Myocardial infarction, angina pectoris or any other cardiac disorder caused by reduced oxygen supply,
Any other clinical condition where oxygen supply is needed, and
Any condition where accelerated wound healing is desired (burn, diabetic ulcer, common wound)
Any condition where the presence of anaerobic bacteria is a major concern.
Eye drops for amelioration or treatment of corneal oxygen deficiency.

For medical indications, the present solutions comprising the water having high content of oxygen, can be administered in any convenient way dependent on the indication to be treated, location of the cells, tissue or organ to be treated etc. Possible delivery methods comprise such as local, oral, intraperitoneal, parenteral, intravasal, delivery methods.

The present solution having a high content of oxygen may also be used in veterinary medicine. As an example, this may be used for treatment or avoidance/reduction of salmon louse in fish farming. The salmon louse is a parasite on salmon which tends to affect salmon in low oxygen conditions. Treatment of salmon with water having high oxygen content is assumed to strengthen the salmon or weaken the salmon louse resulting in reduction or removal of the parasite. According to this aspect, the invention provides a method of treating, avoiding or reducing salmon louse infection in fish, the method comprising treating a salmon with water having a high oxygen content as defined herein.

It is also assumed that the present solutions having a high content of oxygen may be advantageous for cosmetic preparations, to provide an oxygen supply to the skin.

Preparations, medical, veterinary, or cosmetic, may be in the form of a liquid preparation mainly comprising the oxygenated liquid as such, optionally with other conventional additives for the intended use. Alternatively, the oxygenated liquid may be used as an additive to, or for substituting the total liquid phase, in traditional preparations for corresponding use.

The oxygenated liquids may also be used for providing oxygen supply to a compost pile or the like. Adding oxygenated water to the compost will have the same effect on the compost as adding air or oxygen to the compost mass, i.e. making sure that favourable aerobic processes will dominate over less favourable anaerobic processes. There is a risk of setting the compost on fire when adding gaseous oxygen or air into the compost, as the gas will tend to dry out the compost. The combination of dry organic matter, relatively high temperature and oxygen may then result in a fire. Addition of oxygenated water, on the other hand, adds both water and oxygen in a way that facilitates the development of aerobic processes and living organisms, and at the same time adds water to the compost to avoid spontaneous combustion therein.

1. A method comprising using an aqueous solution for culturing cells or tissues or for maintaining cells, tissues or organs, wherein the aqueous solution comprises oxygenated water comprising colloidal minerals, said oxygenated water having an oxygen content of more than 20 mg/L.

2. The method of claim 1, wherein the aqueous solution further comprises solutes for culturing or maintaining viability of the cells, tissues or organs.

3. The method of claim 1, wherein the colloidal minerals are present in said oxygenated water at a level of 10 to 50 ppm.

4. The method of claim 2, wherein said oxygen content is more than 30 mg/L.

5. The method of claim 1, wherein the oxygenated water is obtained by a process comprising:
introducing a pressurized liquid into a piping network to form a flow stream,
adding colloidal minerals to a desired concentration to the flow stream,
injecting gaseous oxygen into the flow stream to produce a mixture of liquid and oxygen bubbles,
providing a linear flow accelerator including a venturi and a magnet adjacent to the venturi and aligned along the venturi for applying a magnetic field of desired field strength across the venturi, and
passing the flowing mixture of liquid and gaseous oxygen bubbles through the linear flow accelerator to accelerate the flowing mixture and to subsequently decelerate the flowing mixture to subsonic speed to break up the gaseous oxygen bubbles.

6. The method of claim 2 for the in vitro culturing of mammalian cells, wherein the oxygenated water comprises at least 10% of the aqueous solution.

7. The method of claim 2, wherein the cells are brain cells, astrocytes, neurons, stem cells and/or acutely dissociated cells.

8. The method of claim 1, wherein the aqueous solution is used for promoting growth of aerobic cells at the expense of anaerobic cells.

9. The method of claim 1, wherein the aqueous solution is used for the in vivo treatment of a tissue where the oxygen supply is impaired, or for treating a deoxygenated tissue.

10. An aqueous cell culture medium comprising oxygenated water and solutes configured to maintain viability of cells, wherein said oxygenated water comprises colloidal minerals and has an oxygen content of more than 20 mg/L.

11. A physiologically acceptable buffer suitable for the in vivo treatment of a tissue where an oxygen supply to the tissue is impaired, wherein the buffer comprises additives and oxygenated water, wherein the additives comprise at least a selected one of glucose, foetal bovine serum (FBS), sodium chloride or potassium chloride, and wherein said oxygenated water comprises colloidal minerals and has an oxygen content of more than 20 mg/L.

12. A method of treatment or amelioration of a medical condition where reduced levels of oxygen in a tissue, organ or the like is implicated, the method comprising administering to a subject in need thereof a preparation comprising an oxygenated liquid comprising colloidal minerals and having a stable oxygen content which is higher than the equilibrium oxygen content of the liquid alone.

13. The method of claim 12, wherein the oxygenated liquid is oxygenated water having an oxygen content of more than 20 mg/L.

14. The method of claim 13, wherein the medical condition is selected from stroke, migraine headache, cerebral hemorrhage, or any other neurological condition where neuroprotective agents are indicated; myocardial infarction, angina pectoris or any other cardiac disorder caused by reduced oxygen supply; corneal oxygen deficiency or any other clinical condition where oxygen supply is needed; a burn, diabetic ulcer or common wound or any other condition where accelerated wound healing is desired; and a condition where the presence of anaerobic bacteria is a major concern.

15. An oxygenated liquid for use in a method of treatment or amelioration of a medical condition where reduced levels of oxygen in a tissue, organ or the like is implicated, wherein said oxygenated liquid comprises colloidal minerals and has a stable oxygen content which is higher than the equilibrium oxygen content of the liquid alone.

16. The oxygenated liquid for use according to claim 15, wherein the oxygenated liquid is oxygenated water having an oxygen content of more than 20 mg/L.

17. The oxygenated liquid for use according to claim 16, wherein the medical condition is selected from stroke, migraine headache, cerebral hemorrhage, or any other neurological condition where neuroprotective agents are indicated; myocardial infarction, angina pectoris or any other cardiac disorder caused by reduced oxygen supply; corneal oxygen deficiency or any other clinical condition where oxygen supply is needed; a burn, diabetic ulcer or common wound or any other condition where accelerated wound healing is desired; and a condition where the presence of anaerobic bacteria is a major concern.

18. A method comprising preparing a medicament for the treatment or amelioration of a medical condition where reduced levels of oxygen in a tissue, organ or the like is implicated, wherein the medicament comprises an oxygenated liquid, wherein said oxygenated liquid comprises colloidal minerals and has a stable oxygen content which is higher than the equilibrium oxygen content of the liquid alone.

19. The method of claim 18, wherein the oxygenated liquid comprises oxygenated water having an oxygen content of more than 20 mg/L.

20. The method of claim 19, wherein the medical condition is selected from stroke, migraine headache, cerebral hemorrhage, or any other neurological condition where neuroprotective agents are indicated; myocardial infarction, angina pectoris or any other cardiac disorder caused by reduced oxygen supply; corneal oxygen deficiency or any other clinical condition where oxygen supply is needed; a burn, diabetic ulcer or common wound or any other condition where accelerated wound healing is desired; and a condition where the presence of anaerobic bacteria is a major concern.

21. An oxygenated liquid for use in therapy, wherein said oxygenated liquid comprises colloidal minerals and has a stable oxygen content which is higher than the equilibrium oxygen content of the liquid alone, preferably wherein said oxygenated liquid is oxygenated water having an oxygen content of more than 30 mg/L and wherein the colloidal minerals are present in said oxygenated water at a level of 10 to 50 ppm.

22. A method of cosmetic treatment, the method comprising administering a preparation to the skin of a subject, wherein the preparation comprises an oxygenated liquid comprising colloidal minerals and having a stable oxygen content which is higher than the equilibrium oxygen content of the liquid alone.

23. The cosmetic treatment method of claim 22, wherein the oxygenated liquid is oxygenated water having an oxygen content of more than 30 mg/L and wherein colloidal minerals are present in said oxygenated water at a level of 10 to 50 ppm.

24. A cosmetic preparation comprising cosmetic constituents and oxygenated liquid comprising colloidal minerals and having a stable oxygen content which is higher than the equilibrium oxygen content of the liquid alone wherein said oxygenated liquid comprises oxygenated water having an oxygen content of more than 30 mg/L and wherein the colloidal minerals are present in said oxygenated water at a level of 10 to 50 ppm.

25. A method comprising using a liquid medium to provide oxygen to cells, tissues or organs, wherein the medium comprises an oxygenated liquid comprising colloidal minerals obtained by a process comprising:
introducing a pressurized liquid into a piping network to form a flow stream,
adding colloidal minerals to a desired concentration to the flow stream,
injecting gaseous oxygen into the flow stream to produce a mixture of liquid and oxygen bubbles,
providing a linear flow accelerator including a venturi and a magnet adjacent to the venturi and aligned along the venturi for applying a magnetic field of desired field strength across the venturi, and
passing the flowing mixture of liquid and gaseous oxygen bubbles through the linear flow accelerator to accelerate the flowing mixture and to subsequently decelerate the flowing mixture to subsonic speed to break up the gaseous oxygen bubbles.

26. The method of claim 25, characterized as a method of treating, avoiding or reducing salmon louse infection in fish, the method comprising treating a salmon with water having an oxygen content of more than 30 mg/L and wherein the colloidal minerals are present in said oxygenated water at a level of 10 to 50 ppm.
